(19) United States
(12) Patent Application Publication
(10) Pub. No.: US 2026/0022146 A1
(43) Pub. Date: Jan. 22, 2026

(54) Treatment for Reducing or Preventing Tissue Fibrosis

(71) Applicant: UNIVERSITY OF NORTH TEXAS HEALTH SCIENCE CENTER AT FORT WORTH, Fort Worth, TX (US)

(72) Inventor: Dimitrios Karamichos, Carrollton, TX (US)

(21) Appl. No.: 18/853,859

(22) PCT Filed: Mar. 30, 2023
(86) PCT No.: PCT/US2023/065149

§ 371 Date: Oct. 4, 2024

Related U.S. Application Data
(60) Provisional application No. 63/328,832, filed on Apr. 8, 2022.

(51) Int. Cl. C07K 14/47; A61K 38/00

(52) U.S. Cl. CPC C07K 14/47; A61K 38/00

(57) ABSTRACT

The present invention includes a method of treating fibrosis in a human patient in need thereof comprising administering to the human patient in need of treatment a therapeutically effective amount of Prolactin-Inducible Protein (PIP) which is capable of inhibiting or reversing the formation of pathogenic fibrils, e g., fibrosis that affects at least one organ selected from the group consisting of skin, intestine, heart, liver, lung, and kidney.

### TECHNICAL FIELD OF THE INVENTION

The present invention relates in general to the field of treatments for reducing or preventing fibrosis, and more particularly, to a novel therapy that reduces and/or prevents fibrosis.

### STATEMENT OF FEDERALLY-FUNDED RESEARCH

None.

### BACKGROUND OF THE INVENTION

Without limiting the scope of the invention, its background is described in connection with tissue fibrosis.

One such treatment for fibrosis is taught in U.S. Pat. No. 10,195,145, issued to Niitsu, et al., entitled “Method for treating fibrosis using siRNA and a retinoid-lipid drug carrier”. This patent is said to teach a method for treating a fibrotic disease by administering a pharmaceutical composition comprising a drug carrier, which comprises a lipid and a retinoid, and a double-stranded nucleic acid molecule, which comprises an antisense sequence to mRNA encoding human hsp47.

Another treatment is taught in U.S. Pat. No. 10,980,789, Nakamura, et al., entitled “Therapeutic agent for fibrosis and inhibitor of nuclear translocation of phosphorylated Smad”. This patent is said to teach a glucosylceramide synthase inhibitor or lactosylceramide synthase inhibitor, or both, that inhibits the nuclear translocation of phosphorylated Smad to treat and prevent fibrosis.

Yet another treatment is taught in U.S. Pat. No. 9,751,923, issued to Duffield, et al., entitled, “Administration of DKK1 muteins to treat fibrosis.” This patent is said to teach molecules, compositions, and methods for treating scarring in organs. The molecules, compositions, and methods treat scarring by modulating the WNT, platelet-derived growth factor receptor (PDGFR), transforming growth factor-beta (TGF3), and/or connective-tissue growth factor (CTGF) signaling pathways.

Despite these advances, a need remains for a therapy that helps reduce existing fibrosis and/or that helps prevent fibril formation from continuing to form, or from forming.

### SUMMARY OF THE INVENTION

As embodied and broadly described herein, an aspect of the present disclosure relates to a method of treating fibrosis in a human patient in need thereof comprising administering to the human patient in need of treatment a therapeutically effective amount of Prolactin-Inducible Protein (PIP) which is capable of inhibiting or reversing formation of pathogenic fibrils. In one aspect, the fibrosis affects at least one organ selected from the group consisting of skin, intestine, heart, liver, lung, and kidney. In another aspect, the human patient suffers from dermal scar formation, skin/dermal fibrosis, keloids, liver fibrosis, heart/cardiac fibrosis, lung fibrosis, kidney fibrosis, pancreatic fibrosis, glomerulosclerosis, lung/pulmonary fibrosis, intestinal fibrosis, interstitial fibrosis, cystic fibrosis of the pancreas and lungs, injection fibrosis, peritoneal fibrosis, endomyocardial fibrosis, mediastinal fibrosis, myelofibrosis, retroperitoneal fibrosis, progressive massive fibrosis, bone fibrosis, fibrosis of vocal cords, a cellular fibrotic phenotype, or nephrogenic systemic fibrosis. In another aspect, the pulmonary fibrosis includes idiopathic pulmonary fibrosis, interstitial lung disease, radiation pneumonitis leading to pulmonary fibrosis; and fibrotic lung disease. In another aspect, the kidney fibrosis is chronic renal failure or diabetic nephropathy. In another aspect, the pancreatic fibrosis is pancreatitis, pancreatic fibrosis, or pancreatic cancer. In another aspect, the cardiac fibrosis is myocardial fibrosis. In another aspect, the skin fibrosis including scleroderma, keloids and hypertrophic scars scleroderma; psoriasis; and Kaposi's sarcoma. In another aspect, the bone fibrosis is selected from the group consisting of myelofibrosis; myeloid leukemia; acute myelogenous leukemia; myelodysplastic syndrome; and myeloproliferative syndrome. In another aspect, the fibrosis is glioblastoma in Li-Fraumeni syndrome or sporadic glioblastoma, gynecological cancer, Hansen's disease, collagenous colitis, or fibrillogenesis. In another aspect, the fibrosis of the vocal cords is selected from the group consisting of vocal cord scarring, vocal cord mucosal fibrosis, and laryngeal fibrosis. In another aspect, the PIP is administered orally, intravenously, topically, intraperitoneally, parenterally, intramuscularly, subcutaneously, or intrapulmonarily. In another aspect, the fibrosis is caused by a trauma or injury selected from burns, cuts, stabs, scrapes, punctures, penetrations, or blunt trauma.

As embodied and broadly described herein, an aspect of the present disclosure relates to a method of treating fibrosis in a patient in need thereof comprising administering to the patient in need of treatment a therapeutically effective amount of Prolactin-Inducible Protein (PIP) which is capable of inhibiting or reversing formation of pathogenic fibrils in skin, intestine, heart, liver, lung, or kidney. In one aspect, the patient suffers from dermal scar formation, skin/dermal fibrosis, keloids, liver fibrosis, heart/cardiac fibrosis, lung fibrosis, kidney fibrosis, pancreatic fibrosis, glomerulosclerosis, lung/pulmonary fibrosis, intestinal fibrosis, interstitial fibrosis, cystic fibrosis of the pancreas and lungs, injection fibrosis, peritoneal fibrosis, endomyocardial fibrosis, mediastinal fibrosis, myelofibrosis, retroperitoneal fibrosis, progressive massive fibrosis, bone fibrosis, fibrosis of vocal cords, a cellular fibrotic phenotype, or nephrogenic systemic fibrosis. In another aspect, the pulmonary fibrosis includes idiopathic pulmonary fibrosis, interstitial lung disease, radiation pneumonitis leading to pulmonary fibrosis; and fibrotic lung disease. In another aspect, the kidney fibrosis is chronic renal failure or diabetic nephropathy. In another aspect, the pancreatic fibrosis is pancreatitis, pancreatic fibrosis, or pancreatic cancer. In another aspect, the cardiac fibrosis is myocardial fibrosis. In another aspect, the skin fibrosis including scleroderma, keloids and hypertrophic scars scleroderma; psoriasis; and Kaposi's sarcoma. In another aspect, the bone fibrosis is selected from the group consisting of myelofibrosis; myeloid leukemia; acute myelogenous leukemia; myelodysplastic syndrome; and myeloproliferative syndrome. In another aspect, the fibrosis is glioblastoma in Li-Fraumeni syndrome or sporadic glioblastoma, gynecological cancer, Hansen's disease, collagenous colitis, or fibrillogenesis. In another aspect, the fibrosis of the vocal cords is selected from the group consisting of vocal cord scarring, vocal cord mucosal fibrosis, and laryngeal fibrosis. In another aspect, the e PIP is administered orally, intravenously, topically, intraperitoneally, parenterally, intramuscularly, subcutaneously, or intrapulmonarily. In another aspect, the fibrosis is caused by a trauma or injury selected from burns, cuts, stabs, scrapes, punctures, penetrations, or blunt trauma. In another aspect, the patient is a human

### BRIEF DESCRIPTION OF THE DRAWINGS

For a more complete understanding of the features and advantages of the present invention, reference is now made to the detailed description of the invention along with the accompanying figures and in which:

FIG. 1 is a graph that shows healthy human primary cells isolated from the Uterus were cultured and stimulated with TGF-β1 only (stimulate fibrosis), PIP only (50, 100, 200, 350, and 500 ng/ml), PIP followed by TGF-β1 (prevention model), or TGF-β1 followed by PIP (rescue model). n=6; *p<0.05; **p<0.01; ***p<0.005; ****p<0.001.

FIG. 2 is a graph that shows healthy human primary cells isolated from the Prostate were cultured and stimulated with TGF-β1 only (stimulate fibrosis), PIP only (50, 100, 200, 350, and 500 ng/ml), PIP followed by TGF-β1 (prevention model), or TGF-β1 followed by PIP (rescue model). n=6; *p<0.05; **p<0.01; ***p<0.005; ****p<0.001.

FIG. 3 is a graph that shows healthy human primary cells isolated from the Lung were cultured and stimulated with TGF-β1 only (stimulate fibrosis), PIP only (50, 100, 200, 350, and 500 ng/ml), PIP followed by TGF-β1 (prevention model), or TGF-β1 followed by PIP (rescue model). n=6; *p<0.05; **p<0.01; ***p<0.005; ****p<0.001.

FIG. 4 is a graph that shows healthy human primary cells isolated from the Heart were cultured and stimulated with TGF-β1 only (stimulate fibrosis), PIP only (50, 100, 200, 350, and 500 ng/ml), PIP followed by TGF-β1 (prevention model), or TGF-β1 followed by PIP (rescue model). n=6; *p<0.05; **p<0.01; ***p<0.005; ****p<0.001.

FIG. 5 is a graph that shows healthy human primary cells isolated from the Skin were cultured and stimulated with TGF-β1 only (stimulate fibrosis), PIP only (50, 100, 200, 350, and 500 ng/ml), PIP followed by TGF-β1 (prevention model), or TGF-β1 followed by PIP (rescue model). n=6; *p<0.05; **p<0.01; ***p<0.005; ****p<0.001.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the terms “treatment” or “therapy” and the like refer to changes in the recipient's status, specifically, with respect to fibrosis or the deposition of fibrils. The changes can be either subjective or objective and can relate to features such as symptoms or signs of the disease or condition being treated. For example, if the patient notes a decrease in fibril deposition, itching, or pain, then a successful treatment has occurred. Conversely, a stabilization or increase in organ function will also demonstrate that a successful treatment has occurred. Similarly, if the clinician notes objective changes, such as by histological analysis of a biopsy sample, then treatment has also been successful. Alternatively, the clinician may note a decrease in lesions or other abnormities upon examination of the patient, which represents an improvement or a successful treatment. Prevention of deterioration of the recipient's status is also included by the term. Therapeutic benefit includes any of a number of subjective or objective factors indicating a response of the condition being treated as discussed herein.

As used herein, a “pharmaceutically effective amount” refers to that amount of an agent effective to produce the intended effect of reducing, preventing and/or fibrosis. Pharmaceutical composition refers to a composition suitable for pharmaceutical use in an animal or animal cell line. The animal may be a mammal, such as a human. A pharmaceutical composition of the invention includes a pharmaceutically effective amount of PIP, which will often also include a pharmaceutically acceptable carrier.

Wound repair is a complex process involving interactions between the wound healing epithelium, a temporary “provisional matrix”, and cells present in the extracellular matrix (ECM). Wounds tend to heal in either a regenerative manner, where the tissue returns to its original state or a fibrotic manner, where a scar is produced. Fibrosis is common to all tissues in the body and is characterized by the overproduction of ECM components, such as cellular fibronectin (cFN), Collagen I (Col I), and Collagen III (Col III), along with the generation of smooth muscle actin (SMA)-expressing cells, termed myofibroblasts. Excessive amounts of ECM materials result in scar formation. In most instances, the process is self-limiting and helps restore the functionality of the tissue(s). The source of the cells that become myofibroblasts is controversial, as both host stromal cells and invading bone marrow-derived cells appear to form the myofibroblast population.

A broad range of chronic diseases, as well as injuries/trauma, can give rise to fibrosis and lead to partial or full tissue/organ failure. Moreover, severe fibrosis is estimated to account for up to 45% of all deaths in the developed world. Thus, there remains an urgent need to understand how fibrosis works and to identify potential therapeutic approaches. The present invention includes methods for reversing and/or preventing fibrosis.

As used herein, the terms “rescue” or “reverse” refers to the ability of a drug to reverse (i.e., rescue) an established fibrotic phenotype (in vitro or in vivo). This can be utilized for any disease/trauma that has already progressed to fibrotic tissue. If you can rescue/reverse fibrosis, that saves people from going to surgical solutions, i.e., corneal transplantation.

As used herein, the terms “prevention” or “inhibition” refers to the ability of a drug to prevent (i.e., arrest) the formation of fibrotic tissue. Certain surgeries are known to cause fibrosis (generally mild versions), so the drug can be given to patients as preventative measure. It can also be used at the very early stages of the fibrotic tissue formation.

Prolactin-induced protein (PIP) is a 17-kDa glycoprotein, originally identified as gross cystic disease fluid protein 15 and a major component of human milk, breast cyst fluid, and saliva. PIP is NCBI Entrez Gene: 5304, and UnitProtKB/Swiss-Prot: P12273, relevant sequences incorporated herein by reference. PIP typically localizes in the cytosol of apocrine epithelia in all major organs and is expressed in the glandular epithelium of seminal vesicles, and as an extra parotid glycoprotein in the sublingual and submandibular glands. Several studies have shown PIP overexpression in both primary and metastatic breast tumors, labeling it as a breast tumor marker.

PIP gene is located on chromosome 7q32-36, has four exons but only one 900 base mRNA transcript. PIP is a 146-amino acid long polypeptide that is found in mammary glands, salivary and lacrimal glands, prostate, and other organs. PIP is shown to have aspartyl protease activity, which signifies the role of PIP as a secreted protein able to mediate ECM degradation. PIP also plays a major role in cell invasion and the viability of apocrine cells and has been shown to play an important role in immunoregulation, by inhibiting T cell apoptosis.

Fibrotic Diseases. Fibrotic diseases are characterized by the excess deposition of fibrous material within the extracellular matrix. These deposits cause changes in tissue architecture that interfere with normal organ function and cause trauma to the affected tissue.

Trauma triggers a wound-healing process and fibrosis is characterized by excessive scarring, including excessive production and deposition of collagen. Thus, normal organ tissues are replaced with scar tissue leading to functional deficiencies, pain, and even organ failure.

There are diverse causes for fibrosis in various organs, e.g., chronic fibrosis occurs as liver cirrhosis, pulmonary fibrosis, sarcoidosis, keloids, kidney fibrosis that are caused by a continuous loss of normal tissue function. By contrast, acute fibrosis is commonly triggered by trauma, such as accidental injuries, infections, surgery, ischemic illness, heart attacks, burns, environmental pollutants, toxins, acute respiratory distress syndrome, radiation, and chemotherapy treatment.

Fibrosis and fibrosis-related pathologies result from the aberrant cross-linking of cellular proteins. Both acute and chronic forms of fibrosis of organs, including all etiological variants of the following: pulmonary fibrosis, interstitial lung disease, fibrotic lung disease, liver fibrosis, cardiac fibrosis, myocardial fibrosis, kidney fibrosis, chronic renal failure, skin fibrosis, scleroderma, keloids, hypertrophic scars, myelofibrosis (bone marrow fibrosis), psoriasis, glioblastoma in Li-Fraumeni syndrome, sporadic glioblastoma, myeloid leukemia, acute myelogenous leukemia, myelodysplastic syndrome, myeloproliferative syndrome, gynecological cancer, Kaposi's sarcoma, Hansen's disease, and collagenous colitis.

The Prolactin-Inducible Protein (PIP) disclosed herein is used to treat fibrotic diseases, including fibrosis that affects at least one organ selected from the group consisting of skin, intestine, heart, liver, lung, and kidney. In certain specific cases the patient suffers from dermal scar formation, skin/dermal fibrosis, keloids, liver fibrosis, heart/cardiac fibrosis, lung fibrosis, kidney fibrosis, pancreatic fibrosis, glomerulosclerosis, lung/pulmonary fibrosis, intestinal fibrosis, interstitial fibrosis, cystic fibrosis of the pancreas and lungs, injection fibrosis, peritoneal fibrosis, endomyocardial fibrosis, mediastinal fibrosis, myelofibrosis, retroperitoneal fibrosis, intestinal fibrosis, progressive massive fibrosis, bone fibrosis, fibrosis of vocal cords, fibrosis of bone, or nephrogenic systemic fibrosis.

Cardiac Fibrosis. Structural remodeling caused by fibrosis of the ventricular wall is a key determinant of clinical outcome in heart disease. Remodeling involves a cycle of production and destruction of extracellular matrix proteins, cell proliferation, and migration, and ultimately apoptotic and necrotic cell death. Cardiac fibroblasts produce growth factors and cytokines that act as autocrine and paracrine factors and secrete extracellular matrix proteins and proteinases.

Kidney Fibrosis includes diabetic nephropathy and chronic renal failure (CRF). Diabetic nephropathy includes glomerulosclerosis and tubulointerstitial fibrosis and is a highly prevalent cause of end-stage renal disease. Diabetic patients constitute the largest population on dialysis, which is very costly, requires multiple weekly multi-hour visits to a clinic, and often requires a kidney transplant. Chronic renal failure is a progressive loss of the ability of the kidneys to excrete wastes, concentrate urine, and recover electrolytes. Chronic renal failure results from any disease that causes gradual loss of kidney function, and fibrosis is the main pathology in CRF.

The two main causes of chronic kidney disease are diabetes and hypertension. However, other factors include acute insults from nephrotoxins, decreased perfusion, hyperlipidemia, hyperphosphatemia, proteinuria, renal ammoniagenesis, among others.

Pulmonary Fibrosis. Interstitial pulmonary fibrosis (IPF) is scarring of the lung caused by a variety of inhaled agents including mineral particles, organic dust, and oxidant gases, or by unknown reasons (idiopathic lung fibrosis). The disease afflicts millions of individuals worldwide, and there are no effective therapeutic approaches. A major reason for the lack of useful treatments is that few of the molecular mechanisms of disease have been defined sufficiently to design appropriate targets for therapy.

Liver Fibrosis. Liver fibrosis is a generally irreversible consequence of hepatic damage such as alcoholic liver disease, biliary disease, viral hepatitis, hemochromatosis, drug-related and cryptogenic cirrhosis, and etiologies of unknown origin. Liver cirrhosis, the end stage of liver fibrosis, often requires liver transplantation and is a leading cause of death in the Western world.

Burns and Scars. A particular problem in fibrotic diseases is the contraction of tissues such as scars. The contraction of tissues including extracellular matrix components, especially of collagen-including tissues, results from many different pathological conditions, including surgical and/or cosmetic procedures. The major components of scar tissue include collagen, extracellular matrix components, and proteins such as elastin that contribute to tissue contraction.

Contraction of collagen and other extracellular matrix components occurs during the healing of burns. The burns may be from the sun, chemical, thermal, or radiation burns of the surface of the skin and the underlying tissues. Burns may also be caused to internal tissues, for example, from exposure to radiation treatments.

Cicatricial contraction can lead to vicious cicatrix, which is a scar that causes deformity. These can result in organ failure, or failure of an organ to operate properly. For example, cicatricial contraction can lead to the obstruction of passages and ducts, known as cicatricial stenosis, such as the uterus. In the digestive tract, e.g., a patient's stomach can be separated into two separate chambers by the contraction of scar tissue formed from stomach ulcers. Contraction of blood vessels may be caused by surgical trauma, such as angioplasty.

It is shown herein that PIP can “rescue” and/or “prevent” fibrotic phenotypes, on several human primary cell types, following induction of fibrosis. It is rather complex to determine how a single target/drug acts in a similar manner on multiple cells/tissues. A potential strategy to overcome this challenge is to target common mechanisms and core pathways that are of central pathophysiological relevance across the different fibrotic diseases. The factors influencing susceptibility to, and initiation of, these diseases are often distinct, with disease-specific and organ-specific risk factors, triggers, and sites of the first injury. Fibrotic remodeling programs with shared fibrotic signaling responses such as transforming growth factor-β (TGFβ), platelet-derived growth factor (PDGF), Wnt, and hedgehog signaling drive disease progression in later stages of fibrotic diseases. The unexpected convergence towards shared responses enables the development of a strategy that is effective across different disease entities and organs.

All the studies were executed with Healthy human primary cells (fibroblasts), purchased from Lifeline® Cell Technology (Frederick, MD), isolated from the Uterus, Prostate, Lung, Heart, and Skin. Cells were expanded and cultured according to the manufacturer protocols. All cells used for the experiments described below were passaged 3 or 4 times, before carrying out the described experiments. Cells were cultured on 2D well plates at 500,000 cells per well, and repeated 6 times (n=6). The cell density was optimized and validated. The duration of all experiments was 4 days, before processing for protein analysis and western blots (WB). For the “rescue” and “prevention” models, the treatment switch between PIP and TGF-β1 happened on day 2 (i.e., half way; equal amount of time per drug). Cells with no treatment served as controls (“dotted-line” on all graphs). Statistical analysis (Prism® software) was performed using ANOVA and/or t-test, as appropriate.

Uterine fibrosis: There is currently no cure for Uterus fibrosis. A patient might receive sex hormone suppression medication in order to slow down the natural production of these hormones, or even be advised to avoid pregnancy. Intriguingly, PIP is known to be regulated by sex hormones (androgens and estrogens). Wide ranges have been reported in both incidence (217-3745 cases per 100000 women-years) and prevalence (4.5-68.6%), depending on study populations and diagnostic methods.

It was found that PIP-only significantly downregulated expression of SMA (FIG. 1; 50 and 100 ng/ml) and cFN (50, 100, 350, and 500 ng/ml). PIP also “rescued” SMA expression (FIG. 1; 200, 350, and 500 ng/ml) and cFN (FIG. 1; 200, 350, and 500 ng/ml). No “prevention” abilities were seen with PIP-stimulation and the Uterus cells. SMA is a marker for fibrosis, as such, if a drug can downregulate SMA, such as PIP, this demonstrates effectiveness. SMA and Collagen Type III are typical markers. cFN is also a good marker but its expression can be more transient.

Prostate fibrosis: It has been speculated that fibrosis of the prostate may increase urethral resistance, and several studies have provided evidence for periurethral fibrosis, decreased elastin, and changes in tissue compliance. The etiology of prostate fibrosis is uncertain, with a current suggestion of the existence of crosstalk between inflammation and fibrosis. There is no cure for the condition, although surgical solutions exist for severe cases. Prostate fibrosis affects about 50 percent of men between the ages of 51 and 60 and up to 90 percent of men older than 80.

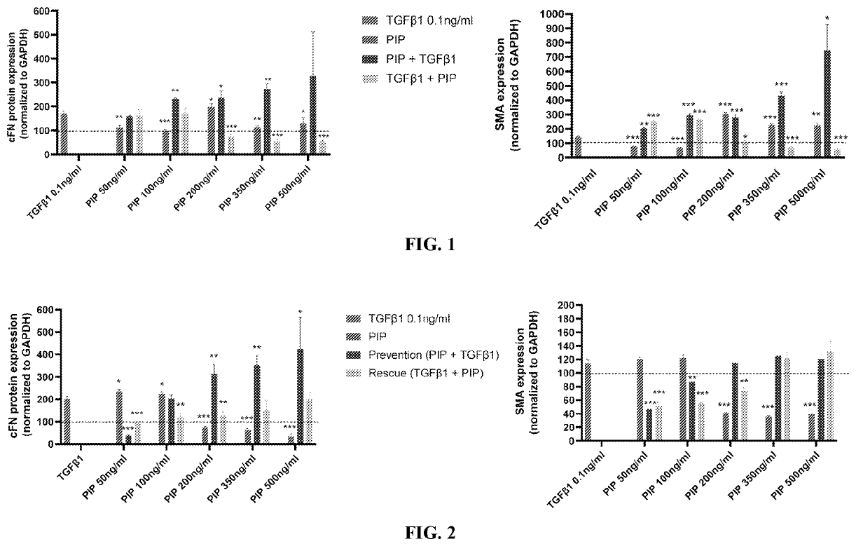

PIP-only significantly downregulated expression of SMA and cFN (FIG. 2; both at 200 and 350, and 500 ng/ml). PIP also “rescued” SMA and cFN expression (FIG. 2; both at 50, 100 and 200 ng/ml). “Prevention” of SMA was achieved with 50 and 100 ng/ml, and cFN with 50 ng/ml of PIP (FIG. 2).

Lung/pulmonary fibrosis: Lung/Pulmonary fibrosis (PF) is a frequently fatal lung disease, and the road to diagnosis can be long and difficult. No one is certain how many people are affected by this condition. Just the idiopathic pulmonary fibrosis (IPF) alone affects more than 50,000 new people each year in the United States.

IPF is only one condition that can cause lung fibrosis. There is currently no cure for IPF. Available treatments aim to prevent progression of lung scarring, relieving symptoms, and helping patients stay active. However, none of those treatments “rescue” lung scarring that has already occurred.

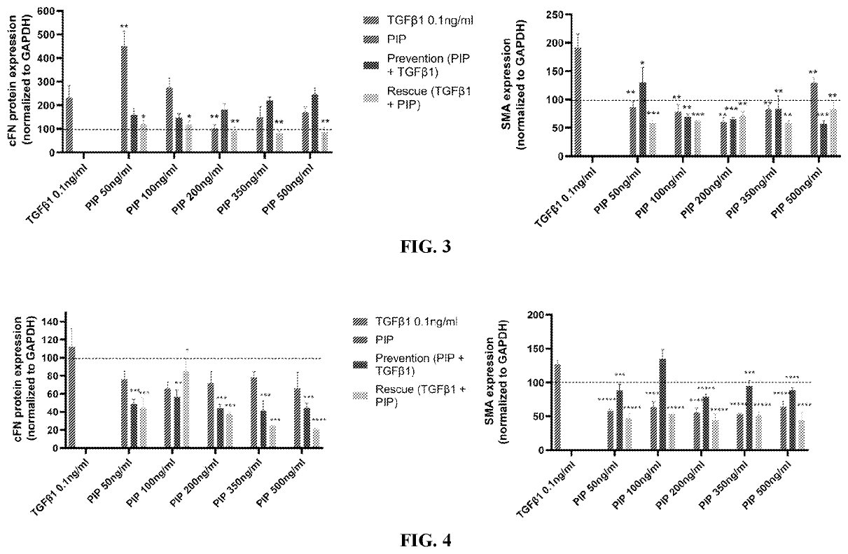

It was found that PIP-only significantly downregulated expression of SMA at all concentrations tested and cFN at 200 ng/ml only (FIG. 3). PIP “rescued” and “prevented” SMA expression at all concentrations, whereas cFN expression was “rescued” at 100, 200, 350, and 500 ng/ml of PIP (FIG. 3). Again, SMA is a marker for fibrosis, as such, if a drug can downregulate SMA, such as PIP, this demonstrates effectiveness. SMA and Collagen Type III are typical markers. cFN is also a good marker but its expression can be more transient.

Heart/cardiac fibrosis: Heart/cardiac fibrosis is a process of pathological ECM remodeling, leading to abnormalities in matrix composition and quality, and impaired heart muscle function. Fibrosis of the cardiac muscle most commonly occur after myocardial infarction; however, various other conditions promote cardiac fibrosis including hypertensive heart disease, diabetic hypertrophic cardiomyopathy and idiopathic dilated cardiomyopathy. Currently therapies to modulate the fibrotic injuries are angiotensin (AT)-converting enzyme and ATI receptor antagonist, a-blockers, endothelin antagonists, statins, and eplerenone. Despite the significant advancements in therapies, the cellular/molecular events that lead to the onset of fibrosis and its process, are still not entirely understood. This is highlighted by the fact that ischemic heart disease and endomyocardial fibrosis are the primary causes of end-stage heart failure.

It was found that PIP-only significantly downregulated expression of SMA but not cFN at all tested concentrations (FIG. 4). PIP, however, “rescued” both SMA and cFN at all concentrations (FIG. 4). Interestingly cFN “prevention” was achieved at all concentrations, and SMA at all but the 100 ng/ml PIP.

Skin/dermal fibrosis: Skin/dermal fibrosis is excessive scarring of the skin, leading to pathologic wound healing response(s). There is a wide spectrum of fibrotic skin diseases: scleroderma, nephrogenic fibrosing dermopathy, mixed connective tissue disease, scleromyxedema, scleredema, and eosinophilic fasciitis. The exact mechanism by which fibrosis develops is not clearly understood, and no proven cures/treatments exist to fully restore a scarred skin.

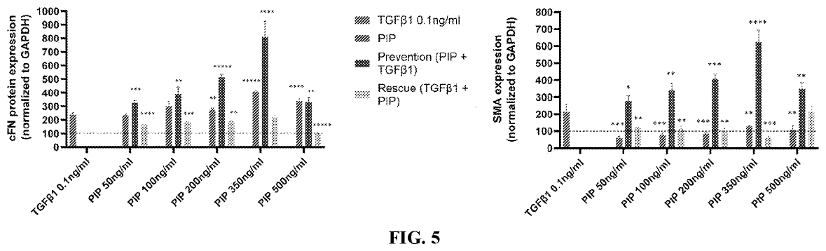

It was found that PIP-only significantly downregulated expression of SMA at all concentrations, but not cFN (FIG. 5). PIP also “rescued” SMA expression (FIG. 5; 50, 100, 200, and 350 ng/ml) and cFN (FIG. 5; 50, 100, 200, and 500 ng/ml). In fact, when cells were stimulated with PIP followed by TGF-β1, both SMA and cFN expressions were significantly upregulated suggesting more fibrotic phenotype.

TABLE 1
Summary of Results.

| | |
|---|---|
| Rescue | Prevention |

| Cell Type | SMA | cFN | SMA | cFN |
|---|---|---|---|---|
| Uterus | + | + | − | − |
| Prostate | + | + | + | + |
| Lung | + | + | + | − |
| Heart | + | + | + | + |
| Skin | + | + | − | − |

Thus, PIP is a new class of drugs to treat fibrotic diseases. The therapeutic role of PIP, in fibrotic diseases, has never been explored or reported before. The present invention allows for fully characterizing target mechanisms and core pathways modulated by PIP. To the inventors' knowledge, this is the first time in any reported study that a single drug has such pronounced effects on multiple fibrotic tissues/organs.

Fibrosis and fibrotic tissue remodeling often lead to organ malfunction significantly affecting function. The medical need for effective antifibrotic therapies is thus very high. A previously proposed strategy, to target multi-organ fibrosis, is to target common mechanisms and pathways that are central across multiple fibrotic diseases. However, such strategy can be extremely difficult given that factors impacting these fibrotic diseases can be organ- or even cell-specific. PIP was originally identified as a breast cancer biomarker, followed by the inventor's discovery that it is a biomarker for Keratoconus; a corneal disease with fibrotic characteristics. The inventors have shown that PIP is modulated by the Transforming growth factor beta (TGF-β) isoforms (-β1, -β2, -β3).

The inventors selected uterus, prostate, lung, heart, and skin, as the initial target systems given their susceptibility to fibrosis. It is shown herein that PIP both “rescued” and/or “prevented” fibrotic markers in vitro.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein. “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA. BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

What is claimed is:

1. A method of treating fibrosis in a human patient in need thereof comprising administering to the human patient in need of treatment a therapeutically effective amount of Prolactin-Inducible Protein (PIP) which is capable of inhibiting or reversing formation of pathogenic fibrils.

2. The method of claim 1, wherein the fibrosis affects at least one organ selected from the group consisting of skin, intestine, heart, liver, lung, and kidney.

3. The method of claim 1, wherein the human patient suffers from dermal scar formation, skin/dermal fibrosis, keloids, liver fibrosis, heart/cardiac fibrosis, lung fibrosis, kidney fibrosis, pancreatic fibrosis, glomerulosclerosis, lung/pulmonary fibrosis, intestinal fibrosis, interstitial fibrosis, cystic fibrosis of the pancreas and lungs, injection fibrosis, peritoneal fibrosis, endomyocardial fibrosis, mediastinal fibrosis, myelofibrosis, retroperitoneal fibrosis, progressive massive fibrosis, bone fibrosis, fibrosis of vocal cords, a cellular fibrotic phenotype, or nephrogenic systemic fibrosis.

4. The method of claim 3, wherein the pulmonary fibrosis includes idiopathic pulmonary fibrosis, interstitial lung disease, radiation pneumonitis leading to pulmonary fibrosis; and fibrotic lung disease.

5. The method of claim 3, wherein the kidney fibrosis is chronic renal failure or diabetic nephropathy.

6. The method of claim 3, wherein the pancreatic fibrosis is pancreatitis, pancreatic fibrosis, or pancreatic cancer.

7. The method of claim 3, wherein the cardiac fibrosis is myocardial fibrosis.

8. The method of claim 3, wherein the skin fibrosis including scleroderma, keloids and hypertrophic scars scleroderma; psoriasis; and Kaposi's sarcoma.

9. The method of claim 3, wherein the bone fibrosis is selected from the group consisting of myelofibrosis; myeloid leukemia; acute myelogenous leukemia; myelodysplastic syndrome; and myeloproliferative syndrome.

10. The method of claim 3, wherein the fibrosis is glioblastoma in Li-Fraumeni syndrome or sporadic glioblastoma, gynecological cancer, Hansen's disease, collagenous colitis, or fibrillogenesis.

11. The method of claim 3, wherein the fibrosis of the vocal cords is selected from the group consisting of vocal cord scarring, vocal cord mucosal fibrosis, and laryngeal fibrosis.

12. The method of claim 1, wherein the PIP is administered orally, intravenously, topically, intraperitoneally, parenterally, intramuscularly, subcutaneously, or intrapulmonarily.

13. The method of claim 1, wherein the fibrosis is caused by a trauma or injury selected from burns, cuts, stabs, scrapes, punctures, penetrations, or blunt trauma.

14. A method of treating fibrosis in a patient in need thereof comprising administering to the patient in need of treatment a therapeutically effective amount of Prolactin-Inducible Protein (PIP) which is capable of inhibiting or reversing formation of pathogenic fibrils in skin, intestine, heart, liver, lung, or kidney.

15. The method of claim 14, wherein the patient suffers from dermal scar formation, skin/dermal fibrosis, keloids, liver fibrosis, heart/cardiac fibrosis, lung fibrosis, kidney fibrosis, pancreatic fibrosis, glomerulosclerosis, lung/pulmonary fibrosis, intestinal fibrosis, interstitial fibrosis, cystic fibrosis of the pancreas and lungs, injection fibrosis, peritoneal fibrosis, endonyocardial fibrosis, mediastinal fibrosis, myelofibrosis, retroperitoneal fibrosis, progressive massive fibrosis, bone fibrosis, fibrosis of vocal cords, a cellular fibrotic phenotype, or nephrogenic systemic fibrosis.

16. The method of claim 15, wherein the pulmonary fibrosis includes idiopathic pulmonary fibrosis, interstitial lung disease, radiation pneumonitis leading to pulmonary fibrosis; and fibrotic lung disease.

17. The method of claim 15, wherein the kidney fibrosis is chronic renal failure or diabetic nephropathy.

18. The method of claim 15, wherein the pancreatic fibrosis is pancreatitis, pancreatic fibrosis, or pancreatic cancer.

19. The method of claim 15, wherein the cardiac fibrosis is myocardial fibrosis.

20. The method of claim 15, wherein the skin fibrosis including scleroderma, keloids and hypertrophic scars scleroderma; psoriasis; and Kaposi's sarcoma.

21. The method of claim 15, wherein the bone fibrosis is selected from the group consisting of myelofibrosis; myeloid leukemia; acute myelogenous leukemia; myelodysplastic syndrome; and myeloproliferative syndrome.

22. The method of claim 15, wherein the fibrosis is glioblastoma in Li-Fraumeni syndrome or sporadic glioblastoma, gynecological cancer, Hansen's disease, collagenous colitis, or fibrillogenesis.

23. The method of claim 15, wherein the fibrosis of the vocal cords is selected from the group consisting of vocal cord scarring, vocal cord mucosal fibrosis, and laryngeal fibrosis.

24. The method of claim 14, wherein the PIP is administered orally, intravenously, topically, intraperitoneally, parenterally, intramuscularly, subcutaneously, or intrapulmonarily.

25. The method of claim 14, wherein the fibrosis is caused by a trauma or injury selected from burns, cuts, stabs, scrapes, punctures, penetrations, or blunt trauma.

26. The method of claim 14, wherein the patient is a human.
